(19) United States
(12) Patent Application Publication
(10) Pub. No.: US 2026/0124307 A1
(43) Pub. Date: May 7, 2026

(54) APPLICATION OF DHA@ZIF-8 IN TREATMENT OF EXPERIMENTAL CEREBRAL MALARIA

(71) Applicants: Hubei University of Medicine, Shiyan City (CN); Tongji University, Shanghai (CN)

(72) Inventors: Jian Li, Shiyan City (CN); Qingfeng Zhang, Shanghai (CN); Jinyu Mo, Shiyan City (CN); Junming Tang, Shiyan City (CN)

(73) Assignees: Hubei University of Medicine, Shiyan City (CN); Tongji University, Shanghai (CN)

(21) Appl. No.: 19/338,011

(22) Filed: Sep. 24, 2025

(30) Foreign Application Priority Data
Nov. 6, 2024 (CN) 202411570868.5

(51) Int. Cl. A61K 47/52; A61K 9/00; A61K 31/357; A61P 33/06

(52) U.S. Cl. CPC A61K 47/52; A61K 9/0019; A61K 31/357; A61P 33/06

(57) ABSTRACT

The disclosure belongs to the technical field of biomedicine, and specifically relates to an application of DHA@ZIF-8 in the treatment of experimental cerebral malaria. In the disclosure, an experimental cerebral malaria model is first established. Subsequently, DHA@ZIF-8 is used for treatment in the model, and the therapeutic efficacy of the nanodrug against experimental cerebral malaria is evaluated using behavioral, pathological, and molecular biological techniques. In the disclosure, the use of ZIF-8 as a carrier achieves the effect of slowly releasing the drug, prolonging the duration of action of the drug in vivo, so as to more effectively kill Plasmodium. Compared with natural administration, this approach extends the duration of action of DHA.

### TECHNICAL FIELD

The disclosure belongs to the technical field of biomedicine, and specifically relates to an application of dihydroartemisinin (DHA) @ zeolitic imidazolate framework-8 (ZIF-8) in the treatment of experimental cerebral malaria (ECM).

### BACKGROUND

CM is a neurology term announced in 2020, which is a brain parasitic disease caused by the direct invasion of Plasmodium, particularly Plasmodium falciparum, and represents one of the most severe types of malaria in patients, with critical condition and high mortality rate. About 1%-2% of falciparum malaria eventually progress to CM, which mainly affects children under the age of five in Africa, with a mortality rate of around 15%-20%. The clinical manifestations of CM include hemiparesis, convulsion, ataxia, coma, disturbance of consciousness, meningeal irritation sign and even death. Even patients who are treated with early and standardized antimalarial chemotherapeutic drugs experience an extremely high fatality rate after the clearance of Plasmodium from their bodies. Furthermore, approximately 25% of survivors suffer from sequelae such as neurological complication and cognitive dysfunction. 10%-20% of surviving children suffer from persistent neurological deficits, cognitive impairment, behavior disorder, and motor function impairment.

Currently, quinine and artemisinin are the recommended drugs for treating CM. Although artemisinin-based drugs have efficient antimalarial effects, the dead parasites after Plasmodium is killed still obstruct local microvessels. The cerebral local ischemic changes and microvascular damage caused by the obstruction fail to be effectively repaired, making the mortality rates caused by CM in children and adults remain as high as 18% and 30%, respectively. Studies have also shown that artemisinin-based combination therapies (ACTs) often fail to protect against cell death, neurological damage and cognitive deficits. Therefore, the use of artemisinin-based drugs alone, particularly DHA, is insufficient to prevent all CM patients from death or neurological dysfunction, suggesting that, on the basis of the existing treatment strategies, it is required to constantly explore novel drug combinations, treatment regimens and adjuvant therapies based on artemisinin.

ZIFs are composed of zinc metal. Zn²⁺ is a tetrahedral structure where four zinc nodes are coordinated with nitrogen atoms of 2-methylimidazole (Hmim) to form ZIF-8. Zinc is the second most abundant metal in the human body, and the imidazole group is present in human histidine, and the cytotoxicity can be negligible. Therefore, ZIF-8 has good biocompatibility and safety. ZIF-8 also has good thermal stability and chemical stability, when dissolved under acidic conditions, it facilitates drug release as needed under the control of pH. Additionally, ZIF-8 has a high specific surface area and porosity, with a flexible structure and adjustable pore size, and can control the release of drug. The premature degradation of drug often hinders therapeutic efficacy. If the drug is adsorbed onto ZIF-8, it can be prevented from premature release. On one hand, the ZIF-8 shell protects the drug, and the sensitivity of drug to the external environment is reduced under the encapsulation by ZIF; on the other hand, the metal ions in ZIF-8 interact with the drug, enabling the transition of drug to a stable isomer and enhancing stability. Drug release occurs in stages: drugs on the ZIF-8 surface dissolve and release first; drugs within the material then gradually diffuse outward due to concentration gradients; and drugs encapsulated within the ZIF-8 cavities are released as the framework collapses, achieving sustained and slow drug release. Adjusting the pore size or modifying the structure of ZIF-8 can alter the affinity between the drug and the nanocarrier, thereby controlling the drug release rate. Modification targeting ligands can deliver drug to lesion sites, achieving precise treatment of diseases. Integrating multiple drugs for combination therapy of CM into a same nanocarrier is a significant research direction for future CM treatment. Therefore, ZIF-8 is considered as a promising nanodrug carrier for CM treatment in the disclosure.

Therefore, in the disclosure, a nanodrug delivery system, DHA@ZIF-8, is constructed; an ECM model is constructed, and the nanodrug is used to intervene, and the therapeutic efficacy of the drug on ECM is evaluated from behavioral and pathological perspectives, aiming to provide an effective solution for the treatment of human CM.

Studies have shown the therapeutic effects of DHA@ZIF-8 in diseases such as tumors. However, its role and mechanisms in infectious diseases, particularly CM, remain unreported. The reason for the insufficient research may be that the use of the nanodrug belongs to interdisciplinary science, involving materials science, pharmacology, and parasitology. Experts in the malaria field primarily focus on the basic problems in molecular biology of Plasmodium pathogenesis, with more attention towards developing new drugs to replace artemisinin or researching vaccines for malaria prevention and treatment through various technical approaches. At present, there are some advancements in the above-mentioned aspects, but there is a certain distance from practical clinical applications. From an interdisciplinary perspective, inspired by nanodrug treatments for major diseases such as tumors, the disclosure conducts an early domestic research on exploring nanodrug treatments for malaria.

Thus, the disclosure aims to elucidate the role and potential mechanisms of DHA@ZIF-8 in the treatment of ECM. Specifically, an ECM model is first established, subsequently, DHA@ZIF-8 is used for treatment in the model, and the therapeutic efficacy of the nanodrug on ECM is observed using behavioral, pathological, and molecular biological techniques.

### SUMMARY

The disclosure first provides an application of DHA@ZIF-8 in the treatment of CM, and the DHA@ZIF-8 is applied in mice.

In some embodiments, the cells that function in vivo are red blood cells of mice infected with Plasmodium berghei.

In some embodiments, the CM is ECM.

In some embodiments, the administration concentration of the DHA@ZIF-8 is defined with 0.5-5 mg/kg of DHA as the final concentration.

In some embodiments, the administration concentration of the DHA@ZIF-8 is defined with 3 mg/kg of DHA as the final concentration.

The disclosure further provides an application of DHA@ZIF-8 in the preparation of a drug for treating CM.

In some embodiments, the in vitro is a cell.

In some embodiments, the CM is ECM.

Compared with the prior art, the disclosure at least has the following beneficial effects.

Given the short in vivo half-life of DHA, the disclosure uses ZIF-8 as a carrier to slowly release the drug, thereby prolonging the duration of action of DHA in vivo, so as to more effectively kill Plasmodium. Compared with natural administration, this approach extends the duration of action of DHA.

### BRIEF DESCRIPTION OF THE DRAWINGS

FIG. 1A shows effects of different drug treatments on weight of ECM mice;

FIG. 1B shows effects of different drug treatments on rapid murine coma and behavioral scale (RMCBS) of ECM mice;

FIG. 1C shows effects of different drug treatments on parasitemia of ECM mice;

and

FIG. 1D shows effects of different drug treatments on survival rate of ECM mice.

FIG. 2A shows representative images of brain tissues after the detection of blood-brain barrier (BBB) permeability in ECM mice following different drug treatments; and

FIG. 2B shows quantitative assessment of BBB permeability after the detection of BBB permeability in ECM mice following different drug treatments.

FIG. 3A shows brain tissues (1000×) stained with hematoxylin and eosin (HE);

FIG. 3B shows liver tissues (400×) stained with HE; and

FIG. 3C shows spleen tissues (400×) stained with HE.

FIG. 4A shows the proportion of liver hemozoin of mice;

FIG. 4B shows the proportion of spleen hemozoin of mice; and

FIG. 4C shows the red pulp (RePu) area in the spleen (400%), where *, P<0.05; **, P<0.01; and ***, P<0.001.

### DETAILED DESCRIPTION

For clearer technical problems to be solved by the disclosure, technical solutions and advantages, detailed description will be provided by reference to the accompanying drawings and specific embodiments.

### Embodiment 1 Material and Method

### Experimental Animal, Plasmodium Strain and Bacterial Strain:

Female C57BL/6 mice (weighing 18-22 g, 8-10 weeks old) were purchased from HNSJA Co., Ltd. in Changsha, China. The mice were raised under the conditions of no specific pathogen and fed with ultraviolet-irradiated feed and purified water to maintain the experimental condition (25±3° C.) with appropriate living and feeding standards. All mice underwent environmental adaptation one week prior to the experiment.

Plasmodium berghei ANKA (PbA) was passaged and cryopreserved in liquid nitrogen for long-term preservation by our laboratory.

The experimental instruments are shown in Table 1.

TABLE 1
Experimental instruments

| | | Production place |
|---|---|---|
| Name | Model | and company |
| Weighing scale | 76175 | Beiximan, China |
| OLYMMPUS | BX53 | Olympus |
| microscope | | corporation, Japan |
| Centrifugal machine | 5424R | Eppendorf, |
| | | Germany |
| Constant-temperature | MK-20 | Allsheng, China |
| metal bath | | |
| Tissue section image | BX53 | Olympus |
| imaging system | | corporation, Japan |
| Micropipette | 0.1-2.5 μL, 3123000217 | Eppendorf, |
| | 0.5-10 μL, 3123000225 | Germany |
| | 2-20 μL, 3123000233 | |
| | 10-100 μL, 3123000241 | |
| | 20-200 μL, 3123000250 | |
| | 100-1000 μL, 3123000268 | |
| Multimode reader | SYNERGY-HT | BioTek, US |
| Mouse tail vein | KW-XXY | Calvin, China |
| injection instrument | | |
| Fridge | −20° C., −80° C. | Haier, China |

The experimental reagents and consumables are shown in Table 2.

TABLE 2
Experimental reagents and kits

| Name | Manufacturer |
|---|---|
| Giemsa dye liquor | Yulu, China |
| DHA | Meryer, China |
| Rapamycin | Meryer, China |
| Atorvastatin | Solarbio, China |
| Syringe | Weigao, China |
| Frozen pipe | Thermo Fisher Scientific, US |
| Glass slide | Citotest, China |
| Methanol | Zhongtian, China |
| Dimethyl sulfoxide (DMSO) | VWR, US |
| Evans Blue (EB) | SIGMA, Germany |
| Formamide | SIGMA, Germany |
| 0.9% normal saline | Kelun, China |
| General-purpose tissue fixative | Absin, China |
| Slide storage box | Beyotime, China |
| PBS buffer powder | Servicebio, UK |
| Pipette tip | Thermo Fisher Scientific, US |

### Embodiment 2

Resuscitation and passage: PbA cryopreserved in liquid nitrogen was placed in a 37° C. water bath; after thawing, the blood was aspirated using a 1 mL syringe and immediately inoculated intraperitoneally into C57BL/6 mice at a dose of 0.2 mL per mouse, and the process was the blood-inoculated resuscitation. When the parasitemia level in the blood-inoculated mice reached 15%-30%, the eyeballs were removed for blood collection, and the blood was placed into a heparin-anticoagulated tube, and was passaged to offspring C57BL/6 mice at a dose of 0.2 mL per mouse according to 1×10⁶ infected red blood cells (iRBCs).

### (1) Construction of an ECM Model

The PbA cryopreserved in liquid nitrogen was taken out and placed in a 37° C. water bath. After thawing, the blood was aspirated using a 1 mL syringe and immediately administered intraperitoneally into C57BL/6 mice at a dose of 0.2 mL per mouse, and the process was blood-inoculated resuscitation. When the parasitemia level in the blood-inoculated mice reached 15%-30%, the eyeballs were removed for blood collection, and the blood was placed into a heparin-anticoagulated tube, and then passaged to offspring C57BL/6 mice at a dose of 0.2 mL per mouse according to 1×10⁶ iRBCs. When the parasitemia level in the offspring mice reached 15%-30%, the eyeballs were removed for blood collection, and the blood was placed into a heparin-anticoagulated tube, and inoculated into experimental mice at a dose of 0.2 mL per mouse according to 1×10⁶ iRBCs, which was designated as day 0 of inoculation. This process was the construction of the ECM model.

### (2) Drug Synthesis

45 mg of zinc nitrate hexahydrate was dissolved in 1.5 mL of deionized water. 99 mg of 2-methylimidazole was dissolved in 2.7 mL of methanol. 5 mg of DHA was dissolved in 0.25 mL of dimethylformamide (DMF). At room temperature, an aqueous solution of zinc nitrate hexahydrate was added to the solutions of 2-methylimidazole and DHA under continuous stirring. The mixture was centrifuged at 12,000 rpm for 8 minutes to obtain DHA@ZIF-8 nanoparticles, which were washed three times with methanol. The DHA@ZIF-8 was synthesized and maintained at 4° C. for later use. The morphological characteristics of DHA@ZIF-8 were examined using a scanning electron microscopy and a transmission electron microscopy. Additionally, the dimension and zeta potential of DHA@ZIF-8 were evaluated using a Malvern laser particle size analyzer. Both the scanning electron microscopy and the transmission electron microscopy show that DHA@ZIF-8 has a typical crystalline structure, with a physical dimension of 250 nm. The zeta potential of DHA@ZIF-8 verified by a Malvern zeta potentiometer is a positive value, and it is worth noting that the value is slightly increased with the loading of DHA. In general, these results indicate that the DHA@ZIF-8 is successfully synthesized.

### (3) Experimental Grouping and Drug Treatment Regimens

On the basis of different drug administration strategies, after adaptive feeding, C57BL/6 mice were weighed and randomly divided into four groups, with 10 mice in each group. DHA, DHA@MOF, and DHA@ZIF-8 were all dissolved in normal saline (0.9% NaCl) containing 5% DMSO, using 3 mg/kg of DHA as the final concentration. Mice in the untreated infection group (PbA) were injected with 5% DMSO on the 3rd day post-infection. 3 mg/kg of DHA was administered to the DHA group on the 3rd day post-infection. 3 mg/kg of DHA@MOF was administered to the MOF group on the 3rd day post-infection. 3 mg/kg of DHA@ZIF-8 was administered to the ZIF group on the 3rd day post-infection. Each drug was administered for five consecutive days, with a single dose of 200 μL per administration.

### (4) Detection of Basic Indicators

Observation of parasitemia: blood was collected from the tail vein to prepare a thin blood smear, and the blood smear was subjected to Giemsa staining. The parasitemia was assessed using the Giemsa-stained thin blood smear under a 100× optical microscope. The parasitemia was examined and quantified by counting the iRBCs among at least 1,000 red blood cells.

Detection of basic behavioral indicators: from day 0 post-infection, the body weight, neurological characteristics, parasitemia, and survival rate of the mice were monitored daily. Neurological characteristics were evaluated using RMCBS, and the evaluation was performed using 10 parameters (fur, limb strength, defensive ability, auricle reflex, toe reflex, contact response, gait, balance, body posture, and exploratory activity). If the mice gradually exhibited the characteristics such as unsteady walking, ataxia, messy fur, no stretching, loss of toe reflex, loss of auricle reflex, convulsion, coma, and even death, the ECM model was successfully constructed.

Assessment of BBB integrity: the protective effect on the brain was evaluated via the BBB integrity. 1% EB dye was diluted in 0.9% NaCl. On the 8th day post-infection, the mice were intravenously injected with 200 μL of 1% EB dye liquor via the tail vein, allowing the dye to circulate for 30 minutes. Subsequently, the mice were anesthetized, and 0.9% NaCl was perfused through the heart into the right atrium until the clear liquid flowed out. The brains were rapidly dissected, photographed, and weighed, then placed in 1.5 mL centrifuge tubes for grinding. Each brain sample was added with 1 mL of formamide and incubated in a constant temperature metal bath of 37° C. for 48 hours. The samples were then centrifuged at 1000 rpm for 10 minutes, and the absorbance was measured at 630 nm using a microplate reader. The EB was quantified on the basis of a standard curve.

Histopathological observation: tissue sampling was performed after the completion of drug administration, that is, on the 8th day post-infection. After euthanasia, brain, liver, spleen, and small intestine tissues were immediately collected and washed three times in cold phosphate-buffered saline (PBS) to remove blood. Subsequently, the tissues were fixed using a general-purpose tissue fixative for 24 hours and embedded in paraffin. Serial sections with a thickness of 4 μm were prepared and stained with HE. Microvascular obstruction and leakage were observed. The sections were observed using an optical microscope, and images were acquired using Olympus cellSens Standard 1.13 software.

### Results

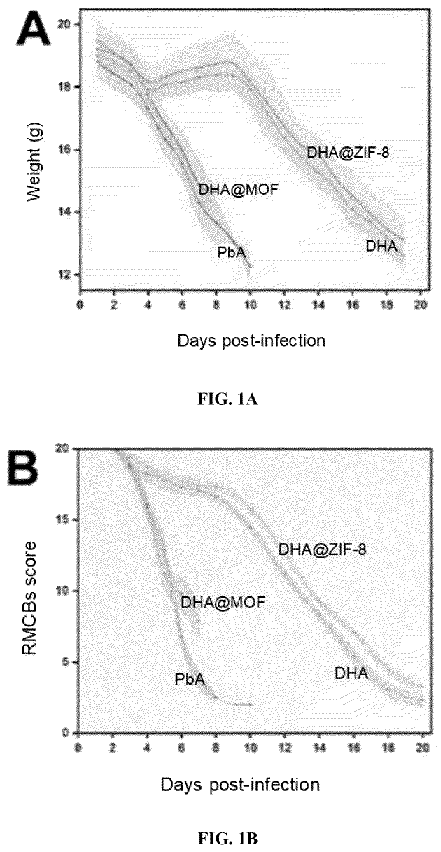

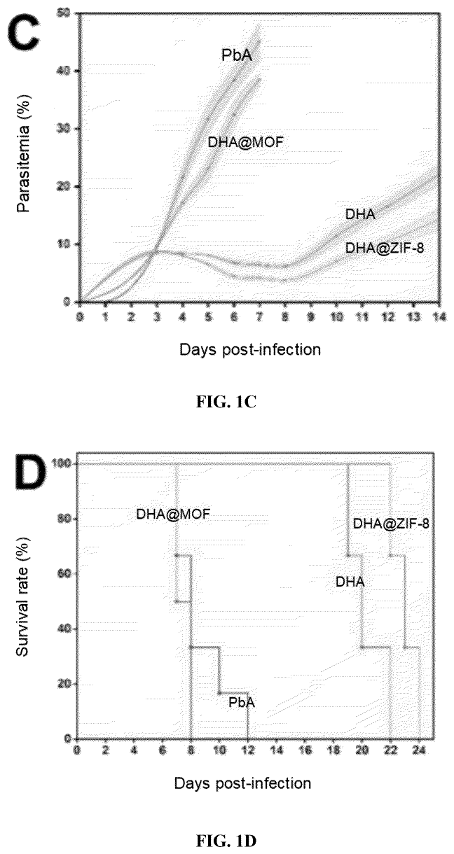

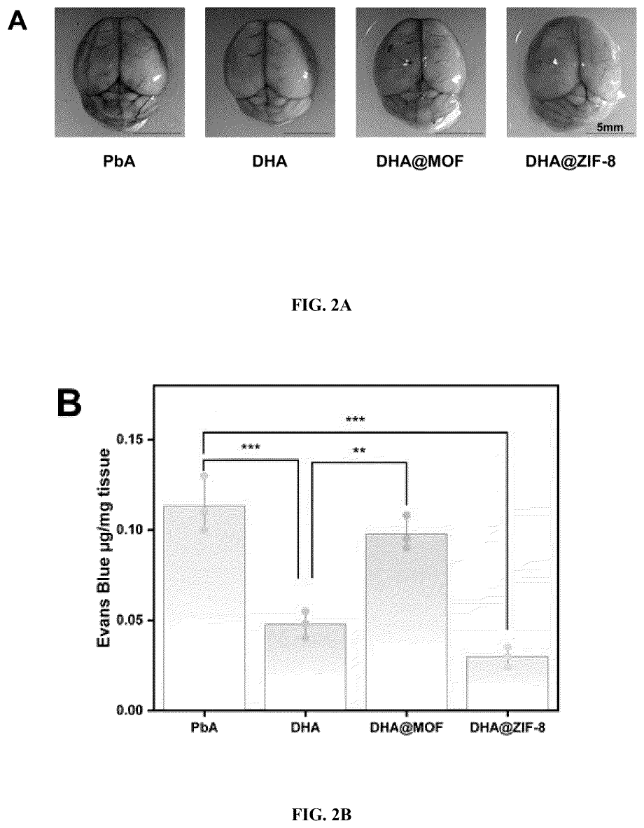

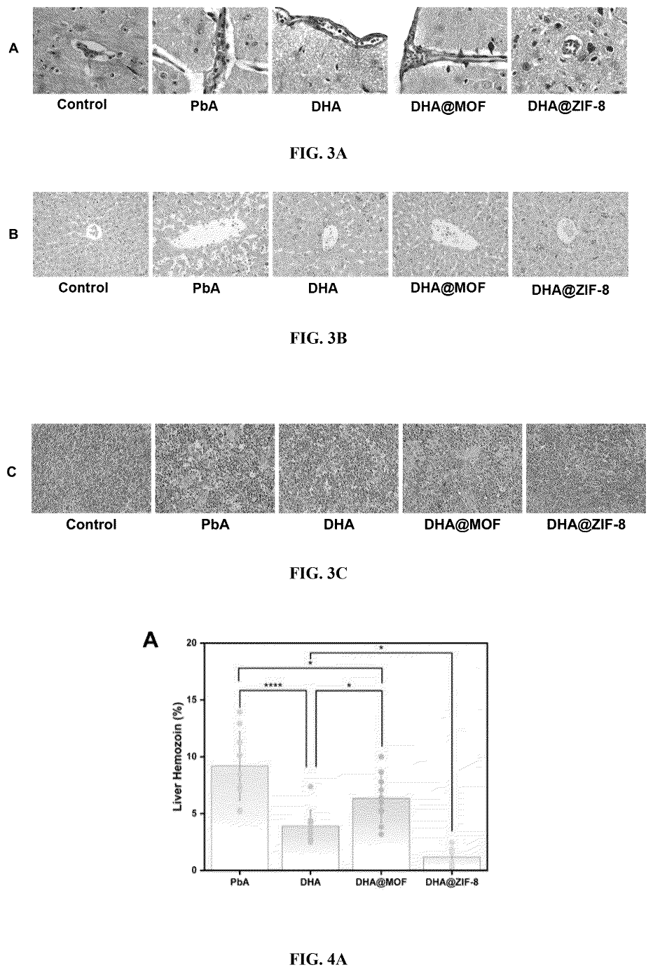

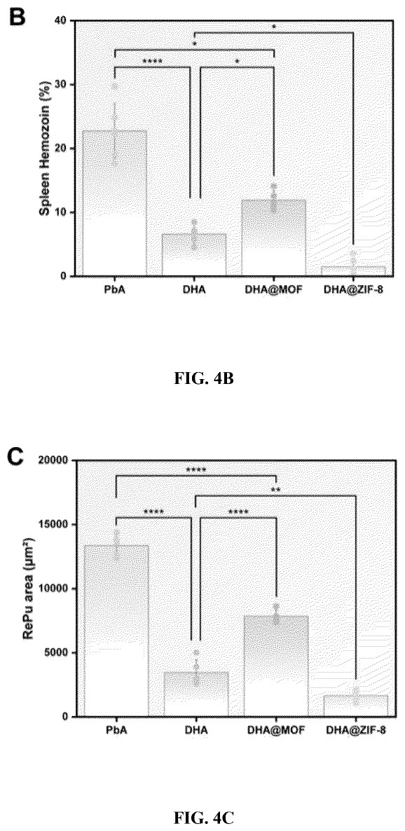

(1) Basic behavioral indicators: PbA-infected mice were respectively treated with DHA, DHA@MOF, and DHA@ZIF-8. The phenotypic behavioral experimental results show that on the 8th day post-infection, the average body weight of mice in the DHA@MOF group significantly exceeds that in the PbA group (P=0.003); on the 18th day post-infection, the body weight of mice in the DHA@ZIF-8 group is the highest, and is greater than that in the DHA group (P=0.047) (FIG. 1A); the RMCBS scores in all groups show a downward trend; the RMCBS scores of the DHA and DHA@ZIF-8 groups show a slight but stable decrease; on the 8th day post-infection, the RMCBS score of mice in the DHA@MOF group is significantly higher than that in the PbA group (P=0.007); the DHA@ZIF-8 group has the highest RMCBS score during the same period; compared with the DHA group, the score of mice in the DHA group is higher (P=0.019) (FIG. 1B); on the 9th day post-infection, in the DHA@ZIF-8 group, the body weight, RMCBS score and survival rate of the mice are the highest, and parasitemia level is the lowest, whereas the DHA@MOF group does not show significant parasiticidal effects (FIG. 1C); all mice in the PbA group die within 12 days, while none of the mice in the DHA@ZIF-8 and DHA groups die within 18 days, and the survival rate of mice in the DHA@ZIF-8 group is significantly higher than that in the DHA group (FIG. 1D). In summary, DHA@ZIF-8 can be considered as a candidate nanodrug delivery system in the treatment of CM by DHA.
(2) Assessment of BBB permeability: visual assessment and EB quantification show significant EB leakage in the PbA group, minimal leakage in the DHA group, and no visible leakage in the DHA@ZIF-8 group (FIG. 2A). In the therapeutic application combining DHA with ZIF-8, there is no statistically significant difference between the DHA group and the DHA@ZIF-8 group in terms of EB leakage (P=0.21) (FIG. 2B). Additionally, the EB level in the DHA@MOF group is higher than that in the DHA@ZIF-8 group (P<0.0001).
(3) Histopathological indicators: the results of brain histopathological analysis show that the DHA@ZIF-8 group is most effective in reducing the sequestration of iRBCs in cerebral capillaries, as well as the adhesion and aggregation of inflammatory cells, suggesting that DHA@ZIF-8 can significantly mitigate cerebrovascular damage and provide significant protection against ECM-induced brain damage (FIG. 3A). Liver histopathological analysis shows that mice in the DHA@ZIF-8 group have the most intact hepatic lobule structure, the most effective reduction in the sequestration of iRBCs in hepatic venous vessels, and almost no hemozoin deposition or adhesion and aggregation of white blood cells, indicating that the DHA@ZIF-8 group has the optimal ability to clear hemozoin (FIG. 3B). Quantitative analysis of hemozoin in the liver is performed (FIG. 4A), revealing the lowest hemozoin content in the DHA@ZIF-8 group. The therapeutic efficacy of the combination of DHA and ZIF-8 on hemozoin metabolism exceeds that of DHA alone (P=0.023). There is significant difference in hemozoin levels between the DHA@MOF group and the PbA group (P=0.016). Histopathological analysis of spleen tissues shows that the DHA@ZIF-8 group has the smallest RePu area, indicating that anemia is mitigated and extramedullary hematopoiesis is reduced, which proves the significant therapeutic efficacy of DHA@ZIF-8 on the spleen (FIG. 3C). Quantitative assessment of hemozoin in the spleen shows the largest RePu area in the PbA group. The RePu area in the DHA@MOF group is significantly reduced compared to that in the PbA group (P<0.0001). Although the RePu area is reduced in the DHA@MOF group, the extramedullary hematopoiesis still presents. The DHA@ZIF-8 group has the lowest hemozoin content (FIG. 4B). There is significant difference between the DHA@ZIF-8 group and the DHA group (P=0.013). Compared with the DHA group, the RePu area in the DHA@ZIF-8 group is significantly reduced (P=0.006), indicating that the extramedullary hematopoiesis is reduced (FIG. 4C).

The foregoing is only preferred embodiments of the disclosure, and it is to be noted that those ordinary skilled in the art may make several improvements and embellishments without departing from the principles of the disclosure, and these improvements and embellishments are considered to be within the scope of protection of the disclosure.

What is claimed is:

1. A method of using DHA@ZIF-8 in the preparation of a drug for treating cerebral malaria, comprising the following steps:
dissolving the DHA@ZIF-8 in a normal saline containing dimethylsulfoxide to obtain the drug, and
injecting the drug to a subject, wherein
the DHA is dihydroartemisinin.

2. The method according to claim 1, wherein the cerebral malaria is experimental cerebral malaria in mice caused by Plasmodium berghei ANKA (PbA) strain.
